(19) United States
(12) Patent Application Publication
(10) Pub. No.: US 2014/0370136 A1
(43) Pub. Date: Dec. 18, 2014

(54) FERMENTED POMEGRANATE OIL COMPOSITION AND METHOD OF PREPARING THE SAME

(71) Applicant: Sung Hag KOO, Chungcheongnam-do (KR)

(72) Inventor: Sung Hag KOO, Chungcheongnam-do (KR)

(21) Appl. No.: 14/081,240

(22) Filed: Nov. 15, 2013

(30) Foreign Application Priority Data
Jun. 12, 2013 (KR) 10-2013-0067212
Jun. 12, 2013 (KR) 10-2013-0067216

(51) Int. Cl. A61K 36/185

(52) U.S. Cl. CPC A61K 36/185
USPC 424/777; 435/134

(57) ABSTRACT

Disclosed are a fermented pomegranate oil composition wherein pomegranates are immersed in edible vegetable oil so that the pomegranates are fermented under anaerobic conditions and active ingredients of the pomegranates are efficiently released to the edible oil to obtain edible vegetable oil containing active ingredients of pomegranates which is useful for cosmetics, functional foods and medicine materials, and a method of preparing the same.

### BACKGROUND OF THE INVENTION

1. Field of the Invention

The present invention relates to a fermented pomegranate oil composition and a method of preparing the same, and more particularly to a fermented pomegranate oil composition and a method of preparing the same wherein pomegranates are immersed in edible vegetable oil so that the pomegranates are fermented under anaerobic conditions and active ingredients of the pomegranates are efficiently released to the edible oil to obtain edible vegetable oil containing active ingredients of pomegranates which is useful for cosmetics, functional foods and medicine materials.

2. Description of the Related Art

Pomegranates are fruits, which originated in Southeast Asia and northwest Indian, and are 6 to 8 cm in diameter, are round-shaped and are surrounded by a hard and yellowish peel. Pomegranate contains about 40% sugar (glucose and fructose) as a main ingredient and about 1.5% citric acid imparting a sour taste thereto as an organic acid. Pomegranate contains a small amount of water-soluble vitamins (B1, B2 and Niacin). In addition, pomegranate skin contains tannin and pomegranate seeds contain natural phytoestrogens, which are beneficial for reduction of menopausal disorders.

Conventional representative compositions using pomegranate have mainly been used as raw materials for cosmetics and oriental medicines for astringents and antidiarrheics (China). In accordance with the traditional Chinese medicine Dictionary, pomegranate leaves, berries, roots and flowers were used for antimicrobial, anthelmintic and anti-viral applications, reduced chance of pregnancy, stimulation of blood clotting, and treatment of chronic diarrhea and dysentery. The North South Donguihak dictionary reads “in fall, pomegranates are harvested and split after fruits are ripe, seeds and the core are discarded and the remaining portion is dried in the sun. Pomegranates taste sour, have warm properties and are poisonous. Pomegranates act on large intestine meridians and nerves, and astringe intestines and stop diarrhea. Pomegranates are used for the treatment of diarrhea and dysentery, atypical uterine bleeding, intestinal bleeding, leukorrhea, ganacratia and proctoptosis and the like, and are administered daily in an amount of 3 to 5 g as a decoction with water or a powder”.

Pomegranates are utilized in a variety of applications, described in Korean Patent No. 1091641 (registered on Jan. 2, 2011, entitled “method of efficiently isolating ellagic acid from pomegranate and functional cosmetics composition with ellagic acid extracts”) which discloses a method of isolating ellagic acid from pomegranate including crushing dried pomegranate, extracting the pomegranate pieces with water and alcohol, macerating the resulting pomegranate pieces for about 6 to 48 hours by a common method such as cold extraction, warm extraction and reflux, and extracting the same one to six times, Korean Patent No. 0562077 (registered on Mar. 10, 2006, entitled “Method for manufacturing Punica granatum L. extract enabling skin protection, skin trouble prevention, inhibition of growth of bacteria detrimental to the skin, acne prevention and antibacterial activity and antibacterial compositions for dermatologically external preparation containing Punica granatum L. extract manufactured by the method”) which discloses a method of preparing a composition by immersing harvested and dried pomegranate in a polar solvent such as ethanol, methanol or acetone at room temperature for 10 to 14 days, followed by extraction and filtration, or heating the resulting pomegranate to 50 to 60° C. for 1 to 5 hours, followed by extraction and filtration, Korean Patent No. 1144059 (registered on May 2, 2012, entitled “Composition for prevention or treatment of diabetes or obesity comprising Punica granatum extract and red ginseng extract”) which discloses preparation of pomegranate extracts using an organic solvent such as alcohol, and Korean Patent Publication No. 10-2012-0092229 (published on Aug. 21, 2012, entitled “Cosmetic composition comprising fermented extracts of tissue cultures of Brassica napus L, Punica granatum and Panax ginseng”, hereinafter, referred to “conventional method”).

The conventional method provides a cosmetic composition which comprises tissue cultures of rape (Brassica napus L), pomegranate (Punica granatum) and wild ginseng, and thus efficiently softens and moistens the skin through anti-aging and anti-wrinkle actions.

However, the conventional method as well as other methods do not demonstrate properties of fermented pomegranates because a composition is prepared by fermenting pomegranate in conjunction with various components, rather than pomegranate alone, using Bacillus genus microbes under dry culture conditions and the composition is contained in only an amount of about 2% by weight in cosmetics and most of the methods relate to acquisition of pomegranate extracts with methanol or ethanol and do not effectively use pomegranates.

Pomegranate is a preparation for external application to the skin which is traditionally known to be effective in treatment of psoriatic skin and seborrheic scalp dermatitis, lice or itching, as well as hypertension, arteriosclerosis and woman menopause disorders. However, compositions having novel efficacy using pomegranate have yet to be developed.

### SUMMARY OF THE INVENTION

Therefore, the present invention has been made in view of the above problems, and it is one object of the present invention to provide a fermented pomegranate oil composition which effectively employs a tannin component and more effectively and variably employs pomegranate based on antibacterial and moisturization effects and a method of preparing the same.

It is another object of the present invention to provide a fermented pomegranate oil composition which exhibits potent efficacy on the treatment of wounds and skin diseases and a method of preparing the same.

In accordance with the present invention, the above and other objects can be accomplished by the provision of a method for preparing a fermented pomegranate oil composition comprising mixing 100 parts by weight of a pomegranate material with 35 to 100 parts by weight of an edible oil, fermenting the resulting mixture of the pomegranate material and the edible oil in a fermenter at a temperature of 15 to 40° C. for 25 to 45 days, and separating the pomegranate material from the fermented mixture.

A vacuum pressure of the inside of the fermenter may be maintained at 0 to 2.5 MPa during fermentation.

The edible oil may be selected from corn oil, soybean oil, olive oil, unpolished rice oil, rapeseed oil, safflower oil, sesame seed oil, perilla oil, camellia oil, green tea oil, sunflower seed oil, grape seed oil, coconut oil and palm oil.

The pomegranate material may be selected from a pomegranate fruit containing no seed, a peel of a dried pomegranate fruit, a bark of pomegranate tree and a seed of pomegranate fruit.

The method may further include mixing 100 parts by weight of a composition of the pomegranate material from the fermented mixture with 70 to 90 parts by weight of a solubilizer (Tween-80), 460 to 500 parts by weight of a concentrated kelp distillate and 5 to 8 parts by weight of lavender oil.

In accordance with another aspect of the present invention, there is provided a method for preparing a fermented pomegranate oil composition comprising mixing 100 parts by weight of a pomegranate material with 35 to 100 parts by weight of an edible oil by immersing the pomegranate material in the edible oil, fermenting the resulting mixture of the pomegranate material and the edible oil in a fermenter at a temperature of 15 to 40° C. for 25 to 45 days, and separating the pomegranate material from the fermented mixture.

A vacuum pressure of the inside of the fermenter may be maintained at 0 to 2.5 MPa during fermentation.

The edible oil may be selected from corn oil, soybean oil, olive oil, unpolished rice oil, rapeseed oil, safflower oil, sesame seed oil, perilla oil, camellia oil, green tea oil, sunflower seed oil, grape seed oil, coconut oil and palm oil.

The pomegranate material may be selected from a pomegranate fruit containing no seed, a peel of dry pomegranate fruit, a bark of pomegranate tree and a seed of pomegranate fruit.

The method may further include mixing 100 parts by weight of a composition of the pomegranate material separated from the fermented mixture with 70 to 90 parts by weight of a solubilizer (Tween-80), 460 to 500 parts by weight of a concentrated kelp distillate and 5 to 8 parts by weight of lavender oil.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a flowchart illustrating a first embodiment of the present invention;

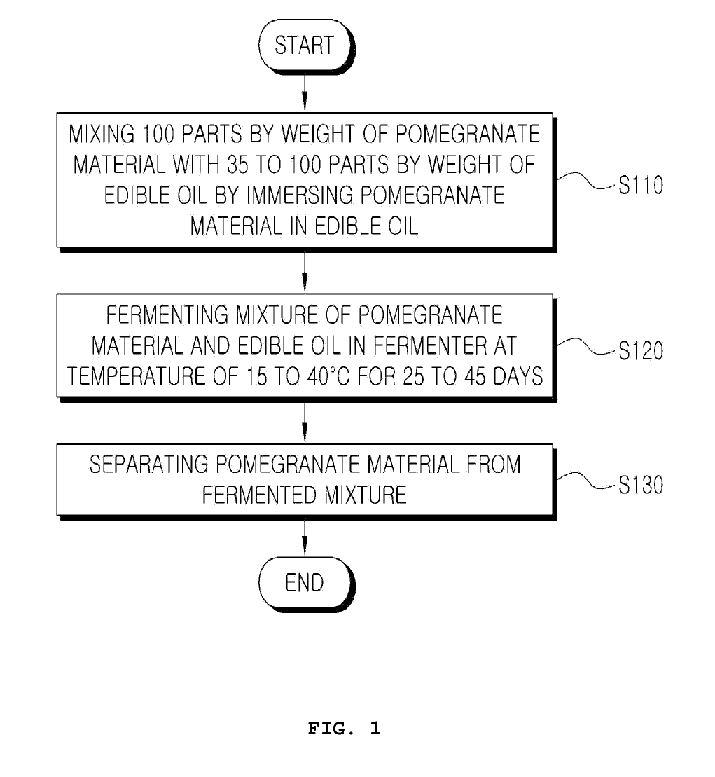

FIG. 2 is a view showing a test report of the fermented pomegranate oil composition according to the present invention by Nutrasource Diagnostics Inc.;

FIG. 3 is a test report of the fermented pomegranate oil composition according to the present invention by the Korea Testing & Research Institute;

FIG. 4A is a graph showing statistics of seborrheic scalp disease patients using the fermented pomegranate oil composition;

FIG. 4B is a graph showing objective statistics of patients using the fermented pomegranate oil composition; and

FIG. 5 is a flowchart illustrating a second embodiment of the present invention.

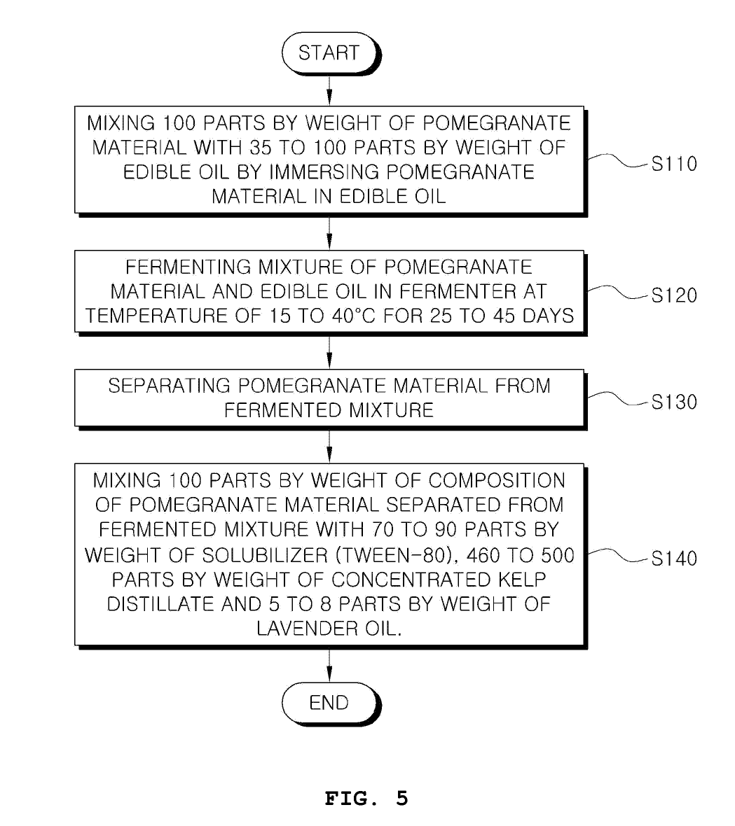

### DETAILED DESCRIPTION OF THE INVENTION

Hereinafter, the fermented pomegranate oil composition and the method of preparing the same according to the present invention will be described in detail.

In accordance with the method of preparing the fermented pomegranate oil composition, first, a pomegranate material is crushed. The crushing process facilitates release of active ingredients of the pomegranate material into the edible oil in a subsequent process of mixing and fermentation with the edible oil.

Pomegranate fruits containing no seeds, peels of dried pomegranate fruits, barks of pomegranate fruits and seeds of pomegranate fruits are selected as byproducts of pomegranate tree containing pomegranate fruits.

The crushed pomegranate material is mixed with the edible oil in a fermenter such that the pomegranate material is sufficiently immersed in edible oil. Regarding a mix ratio, the edible oil is used in an amount of 35 to 100 parts by weight, with respect to 100 parts by weight of the pomegranate material (S110). This mix ratio enables the pomegranate material to be sufficiently immersed in the edible oil. The pomegranate material may decay if exposed to the outside of the edible oil.

In addition, as the mix ratio of the pomegranate material and the edible oil reaches about 1:1, the pomegranate material may be partially exposed to the outside of the edible oil. In this case, the edible oil may be replaced to prevent decay of the pomegranate material.

Any edible vegetable oil may be used as the edible oil without particular limitation, and examples thereof include corn oil, soybean oil, olive oil, unpolished rice oil, rapeseed oil, safflower oil, sesame seed oil, perilla oil, camellia oil, green tea oil, sunflower seed oil, grape seed oil, coconut oil and palm oil. In particular, when the composition according to the present invention is prepared in the form of a capsule, olive oil or corn oil is used, and when the composition is prepared for cosmetic application, olive oil, soybean oil or rapeseed oil is preferably used. When the composition is utilized in cosmetics, the olive oil improves moisturization effects obtained by immersion and fermentation and soybean oil, rapeseed oil, safflower oil, sunflower seed oil, palm oil and grape seed oil do also exhibit the same effects.

In addition, the fermentation is carried out for about 25 to 45 days at room temperature of about 15 to about 40° C. (S120). Accordingly, it is unnecessary to control the temperature or perform heating for fermentation. Acidification of the edible oil can be prevented through fermentation at room temperature and under vacuum. A vacuum pressure of the inside of the fermenter is maintained at 0 to 2.5 MPa during fermentation. As pressure naturally increases due to gas generated by fermentation in the fermenter, the inside of the fermenter is automatically vaccumized and unvaccumized.

As fermentation proceeds, bubbles are generated three days after fermentation and the generation of bubbles is reduced after 25 to 35 days.

Through this process, calcium (Ca), potassium (K), magnesium (Mg), tannin and anthocyanin, which are active ingredients of the pomegranate material, are released into the edible oil, and the pomegranate material is filtered and separated from the fermented mixture (S130).

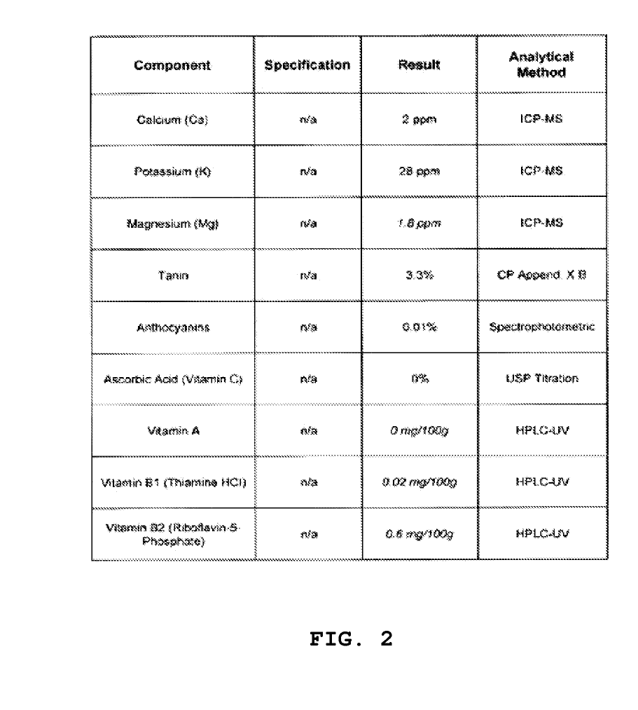

The fermented pomegranate oil composition thus prepared contains 3.3% tannin and is used for preparation of cosmetic products, such as creams, as described in the test report associated with “Pomegranate Oil” by Nutrasource Diagnostics Inc., shown in FIG. 2.

After the fermented pomegranate oil composition is obtained through the aforementioned process, the pomegranate material is crushed, and 30 to 35 parts by weight of a solubilizer is then kneaded with 100 parts by weight of the crushed pomegranate material. The solubilizer may be a natural solubilizer, an olive liquid or soybean lecithin. The kneading enables the composition according to the present invention to be obtained as a sticky cream with natural ingredients containing no chemicals.

The solubilizer contains an oleic fatty acid extracted from caster oil which serves as an emulsifier to prevent isolation of water from essential oil during blending therebetween. The solubilizer is a pale yellow fat-soluble liquid having low viscosity.

In addition, the olive liquid is a solubilizer extracted from olives and the soybean lecithin is a natural surfactant extracted from soybean.

Results of antibacterial test by the Korea Testing & Research Institute demonstrated the fermented pomegranate oil composition prepared in the form of a cream exhibits inhibitory efficacy against Staphylococcus aureus.

In addition, the clinical test which targeted twenty wound or skin disorder patients demonstrates that the composition according to the present invention has potent moisturizing and itching efficacies and healing effects on small wounds or burns when the composition is used for cosmetics or ointments. In particular, the composition efficiently facilitates hemostasis of wounds and inhibits inflammation of wounds. In addition, the composition according to the present invention is effective in the treatment of acne, scaly tetter and psoriasis as well as the treatment of itching and dermatitis such as atopy, and skin sites damaged by ultraviolet light, upon application.

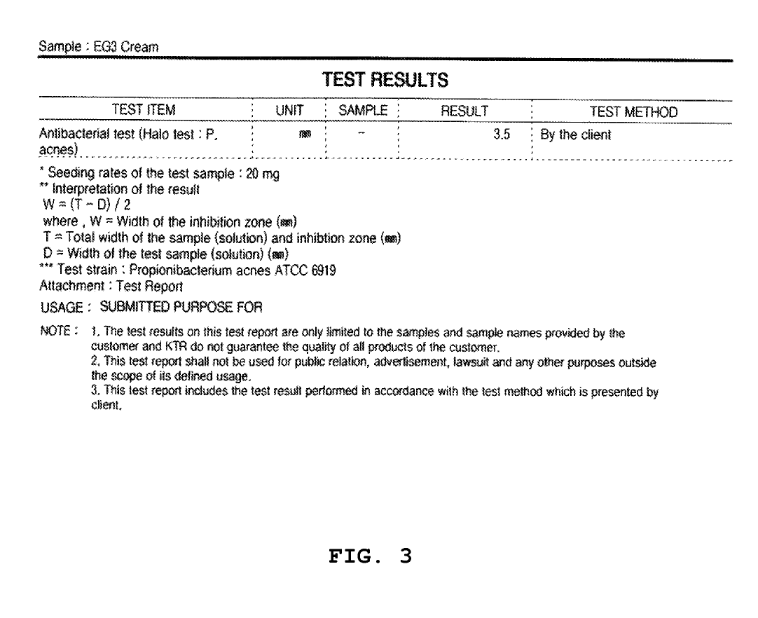

FIG. 3 shows a test report of the fermented pomegranate oil composition according to the present invention obtained by the Korea Testing & Research Institute. In order to identify antibacterial effects of the EG3 cream according to the present invention, the Korea Testing & Research Institute tested antibacterial effects of the EG3 cream against propionibacterium acnes in accordance with a halo test method suggested by the present applicants.

The halo test method is summarized as follows. A prepared plate is homogeneously inoculated with a propionibacterium acne culture using a sterilized cotton swab, a paper disc with a diameter of 6 mm, treated with about 20 mg of the sample according to the present invention, is placed on the plate and is then cultured under anaerobic conditions using an anaerobic jar at 35±1° C. for 72 hours. Then, a diameter of a formed inhibition zone is measured. As a result of measurement of antibacterial properties of the sample according to the present invention under such test conditions, formation of 3.5 mm of the inhibition zone against the propionibacterium acnes used for the test is confirmed.

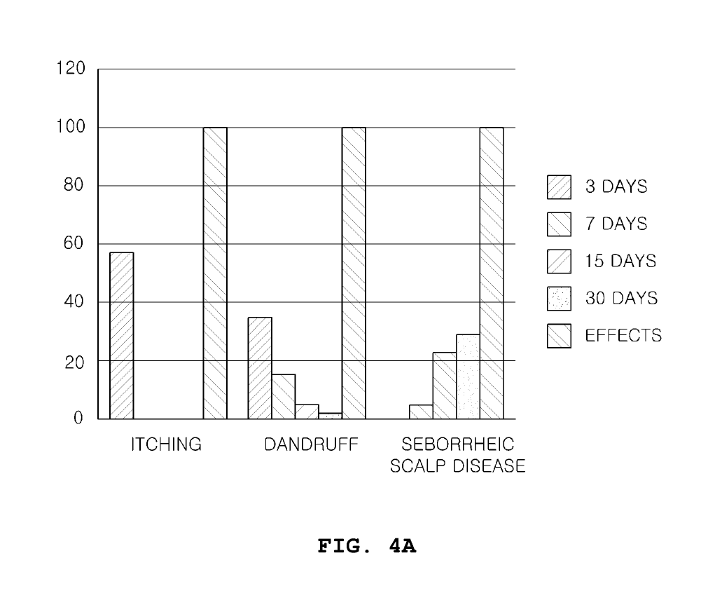

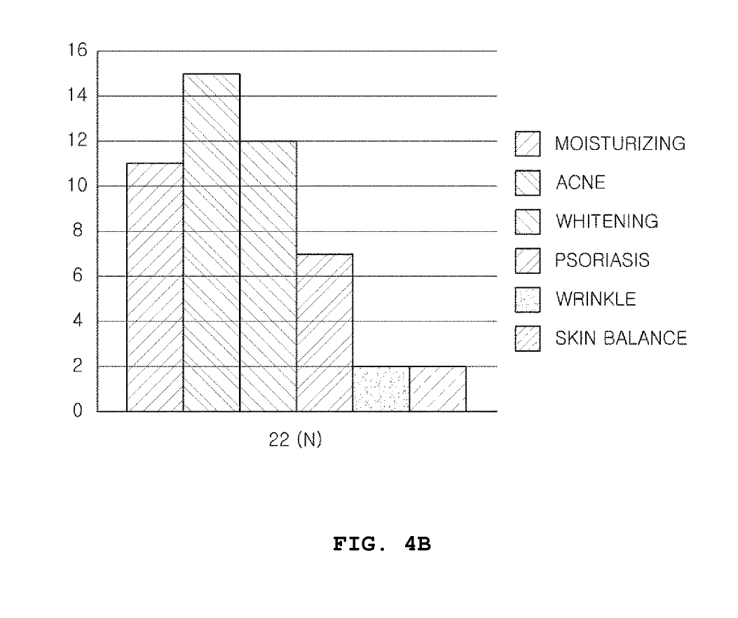

FIG. 4A is a graph showing statistics of seborrheic scalp disease patients using the fermented pomegranate oil composition, wherein the test targets 57 Soonchunhyang university students.

Seborrheic scalp disease symptoms include redness of the scalp, itchiness, generation of dandruff upon scratch with nails, and in serious cases, secretion of discharge, and creation of yellow scabs upon unconsciously repeated scratch of itchy spots. Most known drugs for treatment of seborrheic scalp are steroid ointments, but they do not ultimately remove the cause and only relieve itching. Once seborrheic scalp diseases occur, the diseases gradually become serious and cause alopecia.

57 severe seborrheic scalp disease patients were selected from early to late 20's young male and female volunteers, a substance containing the fermentation pomegranate oil composition according to the present invention was administered to the selected patients and user reviews were monitored for one month, disease severity was checked and the following results were obtained.

TABLE 1

| | | | | | | Recurrence |
|---|---|---|---|---|---|---|
| | | | | | | (after 260 |
| | 3 days | 7 days | 15 days | 30 days | Effects | days) |
| Itching | 57 | | | | 100 | 7 |
| Dandruff | 35 | 15 | 5 | 2 | 100 | 2 |
| Seborrheic | 0 | 5 | 23 | 29 | 100 | 0 |
| scalp | | | | | | |
| disease | | | | | | |

Raw materials used for the composition and contents thereof are 15% of the pomegranate fermentation oil composition, 12% of the solubilizer (Tween-80), 72% of a concentrated kelp distillate and 1% of lavender oil, based on weight.

FIG. 4B is a graph showing objective statistics of patients using the fermented pomegranate oil composition. A cream prepared using the fermentation pomegranate oil composition was tested for severe acne disease volunteers selected from 100 20 to 32 year old male and female volunteers recruited online for two weeks.

The oil composition was prepared in the form of a cream and amounts based on weight of used raw materials, are 35% of purified water, 26% of solubilizer (Tween-80), 22% of fermented pomegranate oil composition, 5% of an olive liquid, 5% of glycerine, 3% of niacin amide, 1.2% of hyaluronic acid, 1% of lavender oil, 1.4% of zinc dioxide and 0.4% of adenosine.

Objective test items associated with use are as follows:

1) Moisturizing property: in an early stage, the skin was glossless, soft and matt due to high viscosity upon application, and after 30 seconds, the cream gradually permeated the skin and the skin was moisturized after 10 minutes. The moisturizing effect was maintained for a long time, in particular, even after 12 hours (excellent moisturizing effect, as compared to conventional products).

2) Removal of acne: a small amount of the cream was applied over the entire surface of the face including acne sites and the face was observed for about one week. As a result, acne was remarkably reduced and acne scars gradually became pale.

3) Whitening: the user of the cream was often told by acquaintances that the dark skin tone generally became clear and brighter. Skin flushing was remarkably relieved after use of the cream.

As a result, the following effects were obtained. Five among the users were continuously tested by questioning. Most of the users underwent good effects and skin improvement effects.

TABLE 2

| | | | | Psoriasis | | Skin |
|---|---|---|---|---|---|---|
| | Moisturizing | Acne | Whitening | (itchiness) | Wrinkle | balance |
| 22 (n) | 11 | 15 | 12 | 7 | 2 | 2 |

For psoriasis patients, seven severe-dry skin (psoriasis) patients used the composition of the present invention and results were then obtained. Severe skin patients refer to those who have unimproved and bad skin although they are treated or have been treated for a long time of 10 years or longer in a general hospital.

Patients who suffered from dead skin cells obtained itching removal and exfoliating effects, when products containing the fermented pomegranate oil composition were applied thereto, and thus continuously used the products. Regarding alopecia prevention effects of shampoo in alopecia patients, a shampoo composition using the fermented pomegranate oil composition was prepared and was tested on 127 severe alopecia patients by the Korean cosmetologist association in Cheonan and Guri for 30 days.

The test mainly targeted ages 35 to 60 and most of the applicants greatly suffered from scalp alopecia due to frequent stress.

First, the composition exhibited excellent treatment effects on scalp itching and dandruff and thus good effects on alopecia.

Ratios of raw materials of the shampoo composition were as follows: fermented pomegranate oil composition 8%; purified water 27%; olive liquid 15%; Ellag-x oil 15%, glycerin 5%, lavender 2%, CDE 10% and cocamidopropyl betaine 18%.

As can be seen from the test, alopecia was considerably reduced as follows:

TABLE 3

| | Number | Average | | | | |
|---|---|---|---|---|---|---|
| | of | of | One | Two | Three | Four |
| Age | subjects | alopecia | week | weeks | weeks | weeks |
| 35~45 | 40 | 80~120 | 70~100 | 65~90 | 30~50 | 5~15 |
| 46~55 | 57 | 60~100 | 45~80 | 40~70 | 25~50 | 10~15 |
| 56~60 | 30 | 65~105 | 60~95 | 55~85 | 20~30 | 5~10 |

The reason for these effects is that conventional shampoos are produced using synthetic chemical surfactants, while the shampoo of the present invention is produced using a smaller amount of natural surfactant.

As apparent from the fore-going, all products are obtained using a remarkably reduced amount of surfactant, thus enabling production of natural products and being advantageously greatly beneficial to health. This is thought to be due to functionality of the fermentation pomegranate oil composition based on great amount of tannin present in pomegranate oil.

The fermented pomegranate oil composition and method of preparing the same according to the present invention exhibit inhibitory effects against Staphylococcus aureus and skin moisturizing effects.

In addition, because the fermented pomegranate oil composition contains a tannin component, the composition administered as a capsule-type preparation prevents fat accumulation, treats hypertension, hyperlipidemia, cerebral apoplexy and the like, has an anti-cancer effect based on antioxidative activity, discharges nicotine from the body, and is effective in relieving inflammation and injuries caused by intestinal hemorrhoids symptoms and treatment of hemorrhoids owing to anti-inflammatory action.

In addition, the fermented pomegranate oil composition has potent efficacy on the treatment of itching as well as small wounds or burn injuries. In particular, the fermented pomegranate oil composition facilitates hemostasis of wounds and inhibits inflammation of wounds.

In addition, the fermented pomegranate oil composition is effective in the treatment of acne, scaly tetter and psoriasis as well as the treatment of itching and dermatosis such as atopy, and skin sites damaged by ultraviolet light, upon application.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A method for preparing a fermented pomegranate oil composition comprising:
mixing 100 parts by weight of a pomegranate material with 35 to 100 parts by weight of an edible oil;
fermenting the resulting mixture of the pomegranate material and the edible oil in a fermenter at a temperature of 15 to 40° C. for 25 to 45 days; and
separating the pomegranate material from the fermented mixture.

2. The method according to claim 1, wherein a vacuum pressure inside the fermenter is maintained at 0 to 2.5 MPa during fermentation.

3. The method according to claim 1, wherein the edible oil is selected from corn oil, soybean oil, olive oil, unpolished rice oil, rapeseed oil, safflower oil, sesame seed oil, perilla oil, camellia oil, green tea oil, sunflower seed oil, grape seed oil, coconut oil and palm oil.

4. The method according to claim 1, wherein the pomegranate material is selected from a pomegranate fruit containing no seed, a peel of dry pomegranate fruit, a bark of pomegranate tree and a seed of pomegranate fruit.

5. The method according to claim 1, further comprising:
mixing 100 parts by weight of a composition of the pomegranate material separated from the fermented mixture with 70 to 90 parts by weight of a solubilizer, 460 to 500 parts by weight of a concentrated kelp distillate and 5 to 8 parts by weight of lavender oil.

6. A fermented pomegranate oil composition prepared by the method according to claim 1.
